(19) United States
(12) Patent Application Publication
(10) Pub. No.: US 2006/0128808 A1
(43) Pub. Date: Jun. 15, 2006

(54) Method of using adapalene in acne maintenance therapy

(75) Inventors: Stephanie Arsonnaud, West Windsor, NJ (US); Pascale Soto, Antibes (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., SOPHIA ANTIPOLIS CEDEX (FR)

(21) Appl. No.: 11/009,024

(22) Filed: Dec. 13, 2004

(51) Int. Cl. A61K 31/192

(52) U.S. Cl. 514/569

(57) ABSTRACT

The present invention provides for a method of using adapalene for the maintenance theray of acne vulgaris to prevent acne recurrence or reduce the severity of the acne recurrence.

### BACKGROUND OF THE INVENTION

1. Field of the Invention

This invention relates to a method of treating acne vulgaris as a maintenance therapy, to prevent acne recurrence or reduce the severity of the acne recurrence.

2. Description of the Related Art

Acne vulgaris is an exceptionally common, recurring disease involving multiple etiological factors including hyperkeratinization, sebaceous gland hyperplasia with seborrhoea, P. acnes proliferation, and inflammation. (See, for example, Thiboutot D. J. Invest Dermatol. 2004; 123:1-12; Pawin H et al. Eur J Dermatol. 2004; 14(1):4-12; and Leyden J J, J Am Acad Dermatol. 2003; 49(3 suppl):S200-S210).

The management of acne can be complex, often requiring aggressive combination therapy and a long-term therapeutic strategy. (See, for example, Thiboutot D. Arch Family Med 2000; 9:179-187; Gollnick H et al, J Am Acad Dermatol. 2003; 49(1 suppl):S1-S37). A recent clinical study investigating the efficacy and safety of adapalene when used concomitantly with oral doxycycline in severe acne subjects showed that the adapalene-doxycycline combination was superior to antibiotic monotherapy, confirming results from previous adapalene-antibiotic combination studies. (See Thiboutot D. et al, Combination therapy with adapalene gel 0.1% and doxycycline for severe acne vulgaris: a multicenter, investigator-blind, randomized, controlled study. Submitted; Wolf J E Jr et al, J Am Acad Dermatol. 2003; 49(3 suppl):S211-S217; Cunliffe W J et al, J Am Acad Dermatol. 2003; 49(3 suppl):S218-S226). Maintenance therapy is necessary for many acne patients, as acne lesions have been shown to return after discontinuing a successful treatment regimen. (See Gollnick H et al, J Am Acad Dermatol. 2003; 49(1 suppl):S1-S37; Thielitz A et al, Br J Dermatol. 2001; 145:19-27). Despite the variety of medications available for the treatment of acute acne, there are few well-controlled studies providing evidence for prophylactic efficacy.

An effective maintenance therapy should prevent acne recurrence by targeting the early stages of comedogenesis and the precursor of mature acne lesions, the microcomedo. (See Gollnick H et al, J Am Acad Dermatol. 2003; 49(1 suppl):S1-S37; Wolf J E. SKINmed. 2004; 3:23-26). Currently, the most effective comedolytic agents are oral isotretinoin and topical retinoids. (See Cunliffe W J, et al, Br J Dermatol. 2000; 142:1084-1091). Oral isotretinoin is an impractical choice for long-term therapy due to the potential for toxicity and teratogenicity. Topical anti-acne medication such as retinoids, could be associated with elevated skin irritation, so careful consideration must be given to the tolerability of a potential maintenance therapy. Cutaneous side effects may decrease the likelihood of treatment adherence, particularly when treating an asymptomatic condition. (See Koo J, SKINmed. 2003; 2:229-33; and Haider A et al, JAMA. 2004; 292:726-735).

Recently published guidelines recommend topical retinoids with or without benzoyl peroxide for maintenance following initial combination treatment with an antimicrobial. (See Gollnick H et al, J Am Acad Dermatol. 2003; 49(1 suppl):S1-S37). Among the currently available topical retinoids, adapalene is considered the best tolerated alone and when used in combination with a variety of topical acne medications. (See Haider A et al, JAMA. 2004; 292:726-735). Adapalene has demonstrated a more favorable tolerability profile than other topical retinoids when applied as monotherapy. (See Dosik J S et al, Cumulative Irritation Potential of adapalene cream and gel, 0.1% compared to tazarotene cream, 0.05% and 0.1%. Cutis. In press; Dosik J S et al, Cumulative irritation potential of adapalene cream and gel, 0.1% compared to tretinoin micro, 0.04% and tretinoin micro 0.1%. Cutis. In press; Greenspan A et al, Cutis. 2003; 72:76-81; Dunlap F E et al, Br J Dermatol. 1998; 139:17-22; Caron D et al, J Am Acad Dermatol. 1997; 36: S110-S112; Egan N et al, Cutis. 2001; 68(suppl 4):20-24; Brand B et al, J Am Acad Dermatol. 2003 September; 49(3 Suppl):S227-S232; Caron D et al, J Am Acad Dermatol. 1997; 36: S113-S115)

### SUMMARY OF THE INVENTION

The present invention provides an effective method of treating acne vulgaris on a long term basis to prevent acne recurrence or to control acne recurrence. The actual invention concerns a maintenance therapy of acne vulgaris. By maintenance therapy we mean: chronic treatment, long term treatment, preventive treatment, reduction of relapse, reduction of severity of relapse, reduction of severity of acne break out.

Generally, the present invention provides a method for preventing acne vulgaris in a patient which comprises first administering adapalene and an antibacterial agent, such as an antibiotic, for at least 12 weeks; and then administering an acne reduction component of adapalene without an antibacterial agent, such as an antibiotic.

The acne vulgaris may also be previously alleviated by any of the methods already known in the art. The present invention provides a method for preventing the recurrence of acne vulgaris in a patient in which the clinical condition associated with acne vulgaris have been alleviated. This method comprises applying to the skin of the patient a topical composition comprising a therapeutically effective amount of adapalene without an antibacterial agent, such as an antibiotic.

More specifically, the present invention provides a method of maintenance therapy which comprises applying to a patient in need which comprises applying to the skin on a regular basis a therapeutically effective amount of a dermatological preparation in the form of an aqueous gel, cream or lotion, said composition comprising a therapeutically effective amount of adapalene without administering an antibacterial agent, such as an antibiotic, to the patient.

The present invention also provides a method of treating a patient already treated for acne by any way applying to the afflicted skin region on a regular basis a therapeutically effective amount of a dermatological preparation in the form of an aqueous gel, cream or lotion, said composition comprising a therapeutically effective amount of adapalene without administering an antibacterial agent, such as an antibiotic, to the patient.

In a particular embodiment, the present invention provides a method of preventing acne vulgaris or treating patients afflicted with acne vulgaris which comprises first orally administering to the patient an effective amount of an antibacterial agent, such as an antibiotic, and topically applying to the afflicted skin region of the patient a therapeutically effective amount of a first dermatological preparation comprising a therapeutically effective amount of adapalene on a regular basis for at least 12 weeks; and afterwards applying to the afflicted skin region on a regular basis a therapeutically effective amount of a second dermatological preparation in the form of an aqueous gel, lotion or cream composition comprising a therapeutically effective amount of adapalene without administering an antibiotic to the patient.

Preferably, the antibiotic is orally administered every day, and the first preparation is applied on a daily basis for the period the preparation is used. Further, it is preferable for the first preparation to be applied for at least 12 weeks. The antibacterial agent that can be used in conjunction with the adapalene may be an antibiotic such as doxycycline, clindamycin, erythomycin, tetracycline, minocycline, trimethroprim, cotrimoxasole, limecycline or benzoyl peroxide.

The second preparation containing adapalene without an antibiotic is preferably applied on a daily basis. Further, it is preferable that the second preparation is applied for at least 16 weeks. Preferably, the second preparation comprises 0.001 to 2% adapalene by weight, most preferably, 0.1% or 0.3% adapalene by weight.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIG. 1 is a schematic flow chart of the disposition of the patients that previously received adapalene-doxycycline combination therapy and who afterwards received either adapalene or vehicle once-daily for 16 weeks.

FIG. 2 is a graphical summary of the maintenance rates for total, inflammatory, and noninflammatory lesion counts at the end of the 16 week period for patients using adapalene gel 0.1% compared with those using the gel vehicle.

FIG. 3 is a graphical summary of median lesion counts of patients using adapalene gel 0.1% compared with those using the gel vehicle at weeks 4, 8, 12, 16 and end-point. FIG. 3(a) shows total lesion count, FIG. 3(b) shows inflammatory lesion count, and FIG. 3(c) shows noninflammatory lesion count.

FIG. 4 shows the tolerability of the treatment by the patients. The various figures show the effects of adapalene vs gel vehicle on mean scores for skin tolerance variables: (a) erythema, (b) scaling, (c) dryness, and (d) stinging/burning at weeks 4, 8, 12 and 16 as well as the worst score during the study. Skin tolerability variables were assessed according to the following scoring scale: none=0, mild=1, moderate=2, and severe=3. Mean scores at each postbaseline visit and worst score (worst observation recorded for a subject during the postbaseline period) are included in the figures.

FIG. 5 summarizes the survey responses from the patients regarding their treatments.

### DETAILED DESCRIPTION OF THE PRESENTLY PREFERRED EMBODIMENTS

The present invention provides for a maintenance treatment of acne vulgaris by an initial treatment with adapalene-doxycycline combination therapy followed by the use of adapalene gel 0.1%. The following details a study that clearly demonstrates the clinical benefit of continued treatment with adapalene gel 0.1% as a maintenance therapy for acne.

A total of 253 subjects that previously received adapalene-doxycycline combination therapy afterwards received either adapalene or vehicle once-daily for 16 weeks. Efficacy and safety criteria included maintenance rate (maintaining at least 50% of improvement from previous study), lesion counts, cutaneous tolerability, and adverse events. Adapalene maintenance therapy resulted in significantly superior maintenance rates (on total lesion: 75% vs 54%; P<0.001) and significantly lower lesion counts (total [P=0.005], inflammatory [P=0.01], and noninflammatory [P=0.02]) compared to vehicle. Adapalene was safe and well tolerated in this study.

### EXAMPLE I

Study Design and Subjects

The efficacy and safety of the adapalene gel 0.1% (Galderma Laboratories, LP, Ft. Worth, Tex.) as a maintenance therapy were compared to gel vehicle in a randomized, multicenter, vehicle-controlled, investigator-blind, parallel group study conducted at 34 centers in the United States between Nov. 13, 2003 and May 25, 2004. This study was designed to set a high threshold for achieving success. Male and female acne subjects, 12 to 30 years of age, who showed at least moderate improvement from baseline (score≦3 on a scale ranging from 0 [clear] to 6 [worse]) after treatment with either adapalene plus doxycycline or doxycycline plus gel vehicle in a previous 12-week study (Thiboutot D et al, Combination therapy with adapalene gel 0.1% and doxycycline for severe acne vulgaris: a multicenter, investigator-blind, randomized, controlled study. Submitted) were enrolled. These subjects were re-randomized consecutively in a 1:1 ratio to receive either adapalene gel 0.1% or adapalene gel vehicle once-daily in the evening for an additional 16 weeks. Subjects were stratified by the treatment assignment received in the previous study using an interactive voice responsive system. The randomization schedule remained blinded from those involved in the clinical conduct of the study. The integrity of the blinding was ensured by packaging the topical medication in identical tubes and requiring a third party other than the investigator/evaluator to dispense the medication.

Exclusion criteria prohibited enrollment of subjects with acne requiring isotretinoin therapy or other dermatologic conditions requiring interfering treatment. Women were excluded if they were pregnant, nursing, or planning a pregnancy as were men with facial hair that would interfere with the assessments. Subjects were provided Cetaphil® daily facial moisturizer SPF 15 (Galderma Laboratories, LP., Ft Worth, Tex.) to use as needed for the symptomatic relief of skin dryness or irritation.

Evaluations for this study occurred at baseline and at weeks 4, 8, 12, and 16. The final visit from the previous 12-week combination study served as the baseline visit for this 16-week maintenance study. A urine pregnancy test was required at baseline and at the final study visit for all females of childbearing potential. Subjects were free to withdraw from the study at any time and for any reason. Subjects not completing the entire study were to be fully evaluated when possible.

This study was conducted in accordance with the ethical principles originating from the Declaration of Helsinki and Good Clinical Practices (GCPs), ICH guidelines, and in compliance with local regulatory requirements. This study was reviewed and approved by an institutional review board. All patients provided their written informed consent prior to entering the study.

Efficacy and Safety Variables

The primary efficacy variable was the failure rate at week 16, defined as the percentage of subjects unable to maintain 50% of total lesion count improvement from the previous 12-week combination therapy study (e.g., a subject entering the maintenance phase after having lost 40 lesions in the combination study was considered as a failure if the lesion count at the end of the maintenance study was increased by more than 20 lesions). Data from this assessment is presented graphically herein as a maintenance rate, which is simply 100% minus the failure rate. Secondary efficacy variables included lesion counts (total, inflammatory and non inflammatory), failure rates for inflammatory and non-inflammatory lesions, as well as global severity and global improvement of the disease. At the last visit, subject satisfaction was assessed via a 5-question survey.

Safety and tolerability were assessed through evaluations of local facial tolerability and adverse events. At each visit, the investigator rated erythema, scaling, dryness, and stinging/burning on a scale ranging from 0 (none) to 3 (severe). Mean scores at each postbaseline visit and worst score (worst observation recorded for a subject during the post-baseline period) were recorded. Adverse events were also evaluated at each visit.

Statistical Analysis

All data analyses were carried out according to a pre-established analysis plan. A sample size of 113 subjects per group was deemed necessary to detect a statistically significant difference in failure rate between treatment groups based on the use of a 2-tailed test with α=0.5 and a power of 90%; an assumption of a 15% efficacy difference between the 2 treatment groups; and a dropout rate of 10%.

Three study populations were analyzed. The safety population was defined as all patients randomized and treated at least once. The intent-to-treat (ITT) population included all randomized subjects who were dispensed study medication. The per-protocol (PP) population included all randomized subjects without any major protocol deviations.

The aim of this study was to show superior efficacy of maintenance therapy with adapalene gel relative to gel vehicle. Analyses for efficacy were performed on week 16 data for the ITT population and the PP population. Last observation carried forward (LOCF) methodology was used to account for missing data for the ITT population analysis (lesion counts). In addition, all subjects with missing data at week 16 were considered failures for the failure rate analysis (worst case). Age was tested at baseline with the analysis of variance (ANOVA) model with treatment, center, and their interaction as factors. All other variables were analyzed by using the Cochran-Mantel-Haenzsel (CMH) test controlling for “analysis center” and previous treatment for both the ITT and PP populations. All tests were 2-sided and used the 0.05 level to declare significance. No adjustment for multiplicity was made.

Results

Subject Disposition and Baseline Characteristics

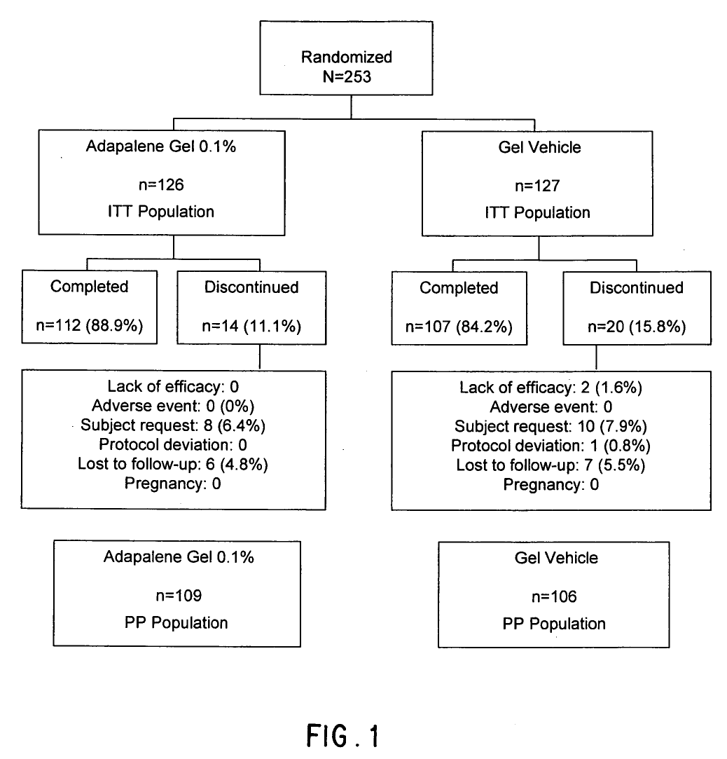

A total of 253 subjects were enrolled in this study and were re-randomized to receive either adapalene gel 0.1% (126 subjects) or gel vehicle (127 subjects; FIG. 1). Subject disposition was similar between the two treatment groups. The per protocol population consisted of a total of 215 subjects (85%) and 219 subjects (87%) completed the study. Discontinuation rates were higher in the vehicle group (15.8%) relative to the adapalene group (11.1%). The most common reason for discontinuation in both groups was subject request (6.4% and 7.9% for the adapalene and vehicle groups, respectively).

Baseline subject characteristics of the ITT population are summarized in Table 1. Demographic characteristics and baseline dermatological scores were comparable between the 2 treatment groups.

Efficacy Evaluation

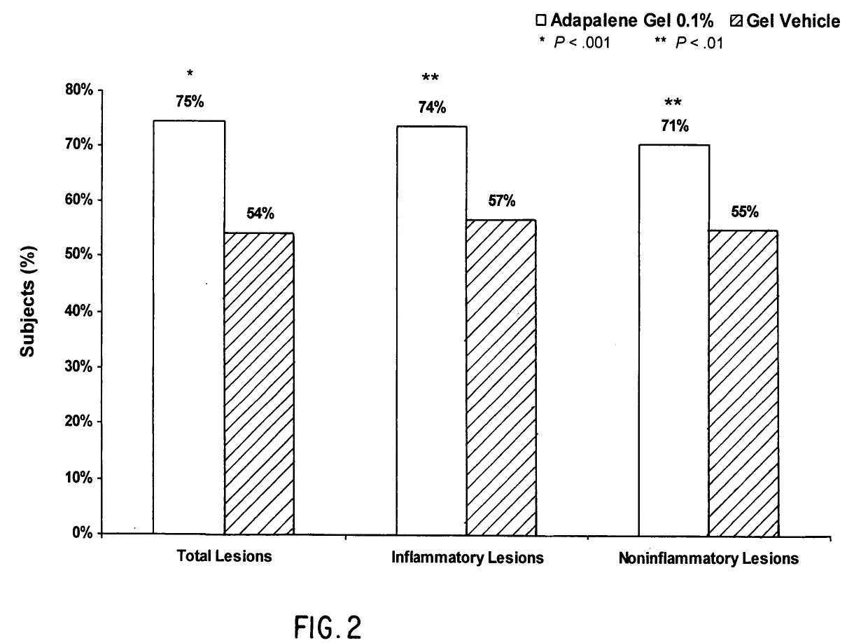

The maintenance rates for total, inflammatory, and noninflammatory lesion counts at end point (week 16, ITT population, worst case) are shown in FIG. 2. These rates reflect the percentage of subjects maintaining at least 50% of improvement from the previous combination study; missing data were treated as failures. Continued treatment with adapalene gel 0.1% resulted in significantly superior maintenance rates in total (75% vs 54%; P<0.001), inflammatory (74% vs 57%; P=0.003), and noninflammatory (71% vs 55%; P=0.007) lesion counts compared to treatment with vehicle (FIG. 2).

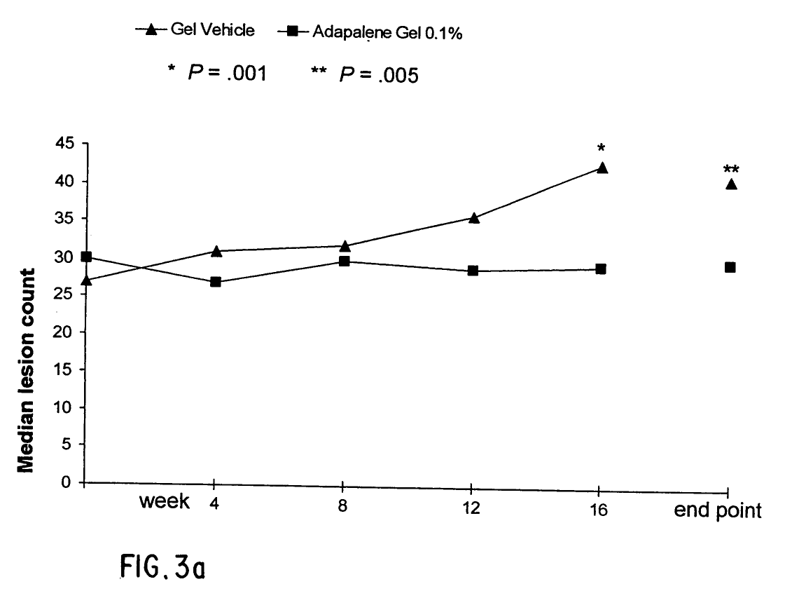

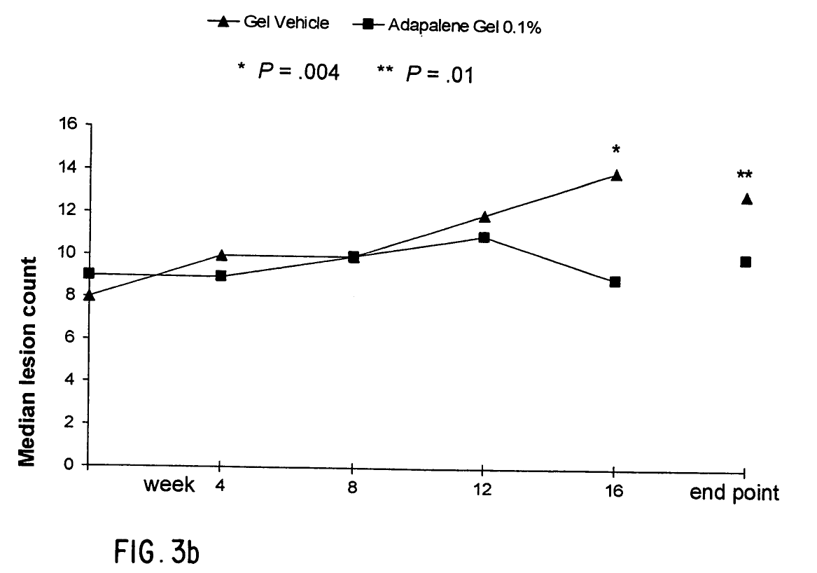

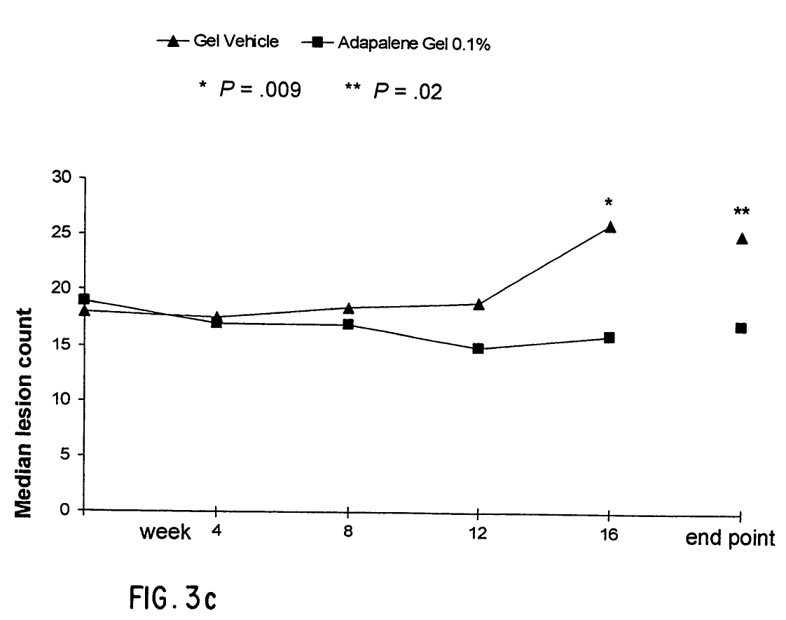

Significantly lower total (P=0.005), inflammatory (P=0.01), and noninflammatory (P=0.02) lesion counts were observed for subjects receiving maintenance therapy with adapalene gel 0.1% relative to vehicle at the study end point (week 16, ITT, LOCF; FIG. 3). During the course of the study, lesion counts for the vehicle group gradually increased from baseline values, while the lesion counts for the adapalene group remained stable or decreased. A numerical difference in total lesion counts between the adapalene and vehicle group is evident beginning as early as week 4, with statistically significant differences observed at week 16 (P=0.001; FIG. 3). Analogous trends were seen for inflammatory (week 16, P=0.004) and noninflammatory (week 16, P=0.009) lesion counts.

Analyzing the full-scale global severity assessment, a significant difference in the distribution of severity scores favoring the adapalene group was observed between the 2 treatment groups (P=0.005), with more subjects “clear” or “almost clear” in the adapalene group relative to the vehicle group (27% vs 16%). Similar efficacy results were obtained in the PP population analysis.

Safety Evaluation

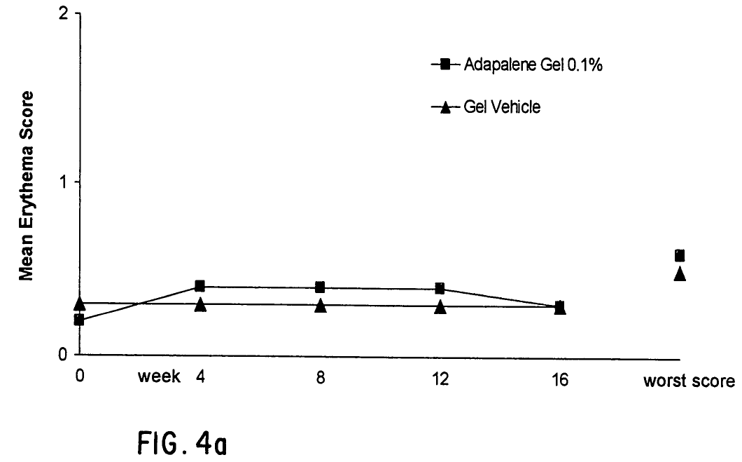

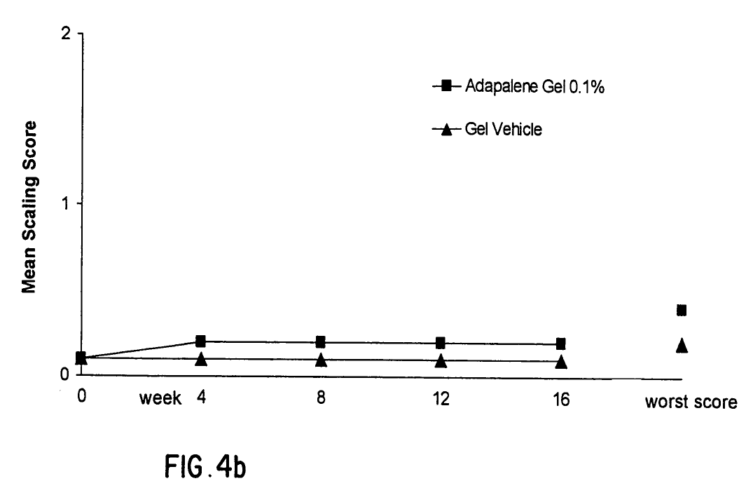

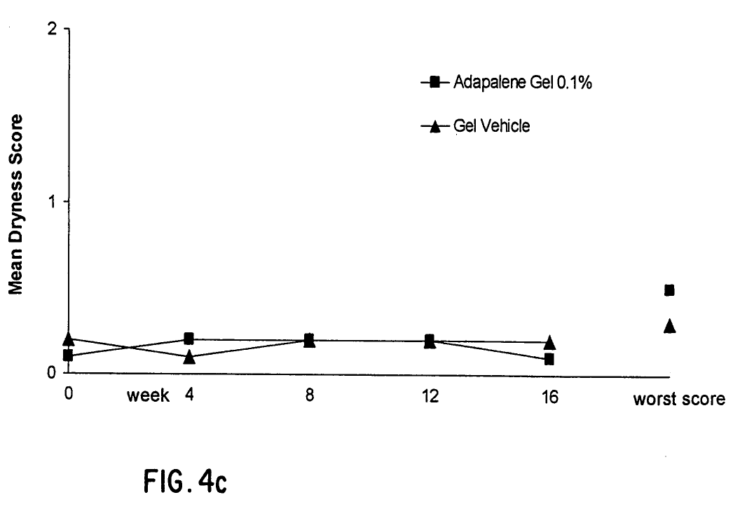

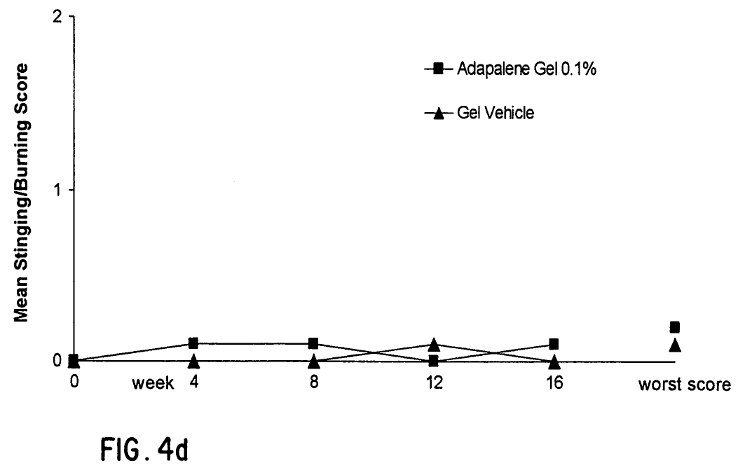

Severity scores for erythema, scaling, dryness, and stinging/burning are summarized graphically in FIG. 4. As expected, local cutaneous tolerability of study treatments was excellent for both groups. Mean tolerability scores for erythema, scaling, dryness, and stinging/burning were less than 1 (mild) for all study visits. Worst scores at any time during the study for these tolerability parameters were all less than 1 (mild) as well. A large majority of subjects in both groups experienced mild or no irritation.

The number of subjects experiencing adverse events was similar in both treatment groups, with 25% and 23% reported for the adapalene and vehicle groups, respectively. During the course of the study, treatment-related adverse events occurred in 3 (2.4%) of adapalene subjects and 1 (0.8%) of vehicle subjects. The most common treatment-related adverse event was pruritus (2 subjects, possibly related). One subject experienced a serious adverse event deemed unrelated to study treatment (suicide attempt by subject with a history of depression). There were no adverse events that led to a study discontinuation and all treatment-related adverse events were mild in severity.

Subject Survey

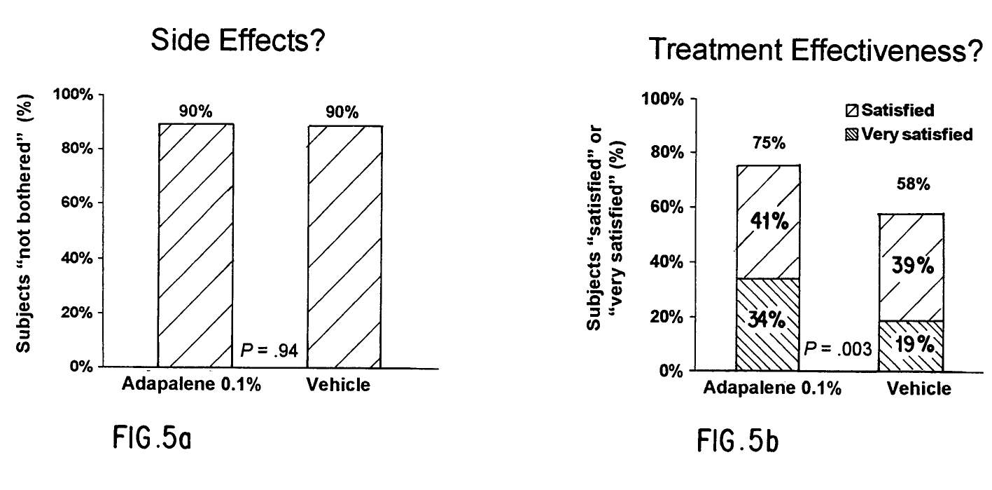

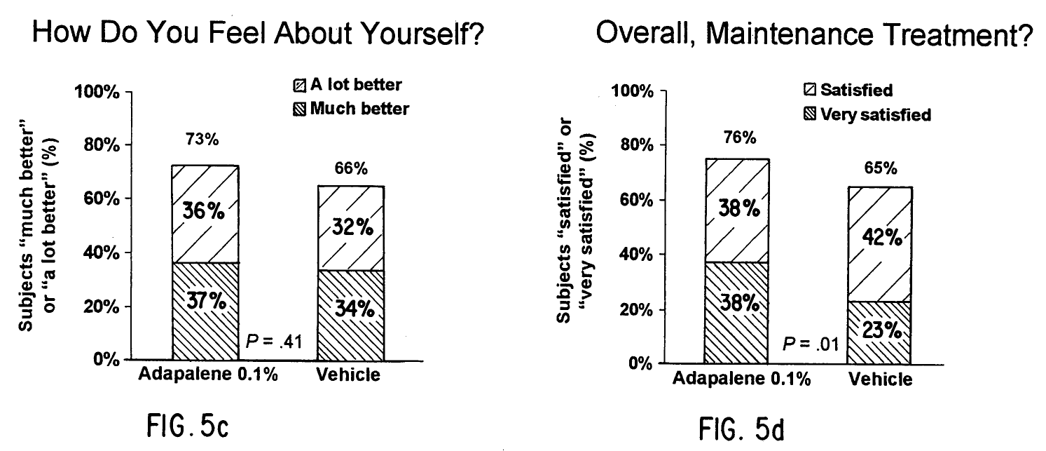

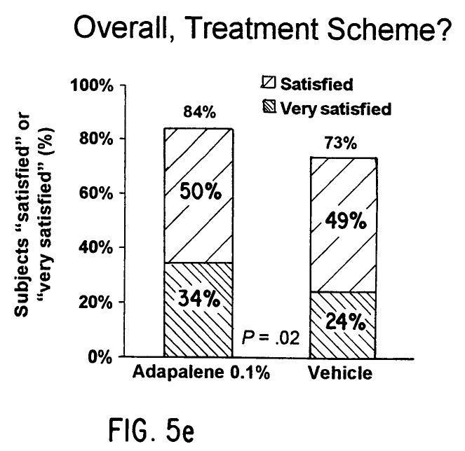

The results from the 5-question survey are illustrated in FIG. 5. A large majority of subjects in both treatment groups were not bothered by side effects (90% adapalene, 90% vehicle; P=0.94). More subjects in the adapalene group felt better about themselves after the study relative to subjects in the vehicle group, although this difference did not reach statistical significance (73% vs 66%; P=0.41). Significantly more subjects in the adapalene group than in the vehicle group were “very satisfied” or “satisfied” with the treatment effectiveness (75% vs 58%; P=0.003) and the overall maintenance treatment (76% vs 65%; P=0.01). Similarly, when subjects were asked how they regarded the overall treatment scheme beginning with the combination therapy, a significantly larger percentage of subjects receiving adapalene maintenance therapy were “very satisfied” or “satisfied” compared to subjects receiving vehicle (84% vs 73%; P=0.02).

Discussion

In light of the chronic nature of acne, maintenance therapy is considered imperative for suppressing the development of subclinical microcomedones and thereby preventing the recurrence of the disease (Gollnick H et al, J Am Acad Dermatol. 2003; 49(1 suppl):S1-S37. Wolf J E, SKINmed. 2004; 3:23-26) Very few well-controlled studies evaluating the clinical benefits of maintenance therapies are available to guide evidence-based decisions for the long-term management of this disease. In an effort to add to our current understanding of the potential of maintenance therapies, this 16-week study evaluated adapalene gel 0.1% as a maintenance therapy in subjects who showed at least moderate improvement in their severe acne in a previous 12-week adapalene-doxycycline combination therapy study. The design of this study set a high threshold for achieving success by utilizing a parallel control group, LOCF/worst case statistical methodology, and re-randomizing subjects after the previous 12-week study.

Overall, results of this study demonstrate a significant clinical benefit of continued adapalene use as a maintenance therapy for acne and underscore the importance of treatment adherence for the success of long-term maintenance therapy. Adapalene provided significantly superior results relative to gel vehicle for all efficacy assessments including total (P=0.005), inflammatory (P=0.01), and noninflammatory lesions counts (P=0.02) as well as the maintenance rate (total lesion: P<0.001). Similarly, significant advantages for adapalene were observed for the global severity assessment (P=0.005) and the global assessment of improvement (P=0.01). Interestingly, a statistically significant difference between adapalene and vehicle was first observed at 4 months, although numerical differences were seen as early as week 4. This observation may reflect the natural life cycle of a comedone, gradually regenerating back to visible acne over several months in the absence of therapy. In addition, combination therapy with a topical retinoid and an antibiotic from the previous study may provide a persistent effect after discontinuing therapy, (Thielitz A et al, Br J Dermatol. 2001; 145:19-27. Thiboutot D, et al, Combination therapy with adapalene gel 0.1% and doxycycline for severe acne vulgaris: a multicenter, investigator-blind, randomized, controlled study. Submitted) which would delay the natural recurrence of acne lesions. The trend of diverging lesion count differences between the adapalene and vehicle groups suggests that a continued benefit may be obtained beyond 4 months; however, additional studies of longer duration would be necessary to confirm this observation.

As expected from previous studies, adapalene was safe and well tolerated. Only 3 (2.4%) subjects receiving adapalene experienced treatment-related adverse events and the mean worst score for each of the local cutaneous tolerability variables was none or mild for a large majority of adapalene subjects. In addition, results from the subject satisfaction questionnaire support the physician assessments, as subjects indicated that the side effects did not bother them and overall, they had significantly greater satisfaction with adapalene maintenance therapy (P=01).

This is the first rigorously controlled study evaluating a topical retinoid as a maintenance treatment for acne. Although no literature documenting other topical retinoid maintenance studies are available for comparisons, the results are consistent with the known comedolytic properties as well as the well-documented safety profile of adapalene (Haider A et al, JAMA. 2004; 292:726-735. Cunliffe W J et al, Br J Dermatol. 1998; 139:48-56. Waugh J et al, Drugs. 2004; 64:1465-1478). In addition to efficacy, adapalene fulfills 3 important requirements of a maintenance therapy (Wolf J E SKINmed. 2004; 3:23-26). First, topical retinoids target comedogenesis, normalizing the altered pattern of follicular keratinization and minimizing the formation of new acne lesions (Gollnick H et al, J Am Acad Dermatol. 2003; 49(1 suppl):S1-S37). The lipophilicity of adapalene allows for penetration directly to the site of microcomedo formation, the lipid-rich pilosebaceous unit (Shroot B et al, J Am Acad Dermatol. 1997; 36(2 suppl):S96-S103. Allec J et al, J Am Acad Dermatol. 1997; 36(2 suppl):S119-S125). Second, adapalene is regarded as the best tolerated topical retinoid, (Haider A et al, JAMA. 2004; 292:726-735) consistently demonstrating a more favorable cutaneous irritation profile than other topical retinoids, including all tazarotene (Dosik J S et al. Cumulative Irritation Potential of adapalene cream and gel, 0.1% compared to tazarotene cream, 0.05% and 0.1%. Cutis. In press. Greenspan A et al, Cutis. 2003; 72:76-81 and tretinoin formulations. Dosik J S et al, Cumulative irritation potential of adapalene cream and gel, 0.1% compared to tretinoin micro, 0.04% and tretinoin micro 0.1%. Cutis. In press. Greenspan A, et al, Cutis. 2003; 72:76-81. Dunlap FE et al, Br J Dermatol. 1998; 139:17-22. Caron D et al, J Am Acad Dermatol. 1997; 36: S110-S112. Egan N et al, Cutis. 2001; 68(suppl 4):20-24). Tolerability is an essential trait for a successful maintenance regimen, as the low potential for skin irritation improves the likelihood of treatment adherence (Koo J. SKINmed. 2003; 2:229-33). Finally, convenience is also critical for ensuring patient adherence to therapy. Adapalene gel can be applied once-a-day immediately after washing and therefore can be easily integrated into a patients' daily routine (Dunlap F E, et al, Br J Dermatol. 1998; 139(suppl 52):23-25).

Taken together, the cumulative results of this maintenance study and the preceding 12-week adapalene-doxycycline combination therapy study support the recommendations of the recently published consensus guidelines for acne (Gollnick H, et al. Management of acne. J Am Acad Dermatol. 2003; 49(1 suppl):S1-S37). The report states that an effective strategy for moderate to severe acne is to utilize combination therapy at the onset of therapy with both a topical retinoid and an antibiotic (oral or topical) until reasonable clearing has occurred. Then, the antibiotic therapy should be discontinued to reduce the potential for developing resistance and the topical retinoid continued to prevent recurrence. These 2 studies provide an instructive illustration of this practice recommendation over a total of 7 months

In summary, this study clearly demonstrates the clinical benefit of continued treatment with adapalene gel 0.1% as a maintenance therapy following combination therapy with an antimicrobial. Therefore, adapalene should also be used for the long-term management of this disease to ensure acne lesions remain in remission.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

### Table 1

APPLN. FILING DATE: Dec. 13, 2004

TITLE: METHOD OF USING ADAPALENE IN ACNE MAINTENANCE THERAPY

INVENTOR(S): STEPHANIE ARSONNAUD ET AL.

APPLN. DOCKET NO.: 034227-714 SHEET 1 OF 1

TABLE 1
Subject demographics and baseline
characteristics (ITT population)

| | | Adapalene | | | | |
|---|---|---|---|---|---|---|
| | | Gel 0.1% | | Gel Vehicle | | P |
| Demographic Parameter | | (n = 126) | | (n = 127) | | value^{a} |
| Age (years) | Mean (SD) | 18.1 | (4.2) | 17.8 | (3.9) | 0.61 |
| | Range | 12 to 30 | | 12 to 32 | | |
| Gender | | | | | | 0..34 |
| Male | n (%) | 65 | (51.6) | 73 | (57.5) | |
| Female | n (%) | 61 | (48.4) | 54 | (42.5) | |
| Race | | | | | | 0.56 |
| Caucasian | n (%) | 74 | (58.7) | 76 | (59.8) | |
| Black | n (%) | 17 | (13.5) | 14 | (11.0) | |
| Asian | n (%) | 6 | (4.8) | 3 | (2.6) | |
| Hispanic | n (%) | 29 | (23.0) | 32 | (25.2) | |
| Other | n (%) | 0 | (0) | 2 | (1.6) | |
| Lesion Counts | | | | | | |
| Total | Mean (SD) | 32.7 | (23.1) | 33.2 | (22.9) | 0.98 |
| Inflammatory | Mean (SD) | 9.7 | (7.2) | 10.2 | (8.3) | 0.73 |
| Noninflam- | Mean (SD) | 22.9 | (19.7) | 22.9 | (19.0) | 0.88 |
| matory | | | | | | |
| Global | Clear | 5 | (4.0%) | 2 | (1.6%) | 0.69 |
| Severity | | | | | | |
| Assessment | Minimal | 24 | (19.1%) | 26 | (20.5%) | |
| | Mild | 64 | (50.8%) | 61 | (48.0%) | |
| | Moderate | 33 | (26.2%) | 38 | (29.9%) | |
| | Severe | 0 | (0.0%) | 0 | (0.0%) | |
| | Very | 0 | (0.0%) | 0 | (0.0%) | |
| | Severe | | | | | |

1. A regime/regimen for acne maintenance therapy applied to a patient in need thereof which comprises applying to the skin on a regular basis a therapeutically effective amount of a dermatological composition, said composition comprising a therapeutically effective amount of adapalene and a dermatological vehicle formulated as an aqueous gel, cream or lotion, without administering an antibacterial agent to the patient.

2. A regime/regimen for treating a patient already treated for acne by any way, which comprises applying to the afflicted skin region on a regular basis a therapeutically effective amount of a dermatological composition, said composition comprising a therapeutically effective amount of adapalene and a dermatological vehicle formulated as an aqueous gel, cream or lotion, without administering an antibacterial agent to the patient.

3. The regime/regimen of claim 1 or 2 wherein said composition is administered every day.

4. The regime/regimen of claim 1 or 2 wherein said composition is applied daily or every other day.

5. The regime/regimen of claim 1 or 2 wherein said composition is applied for at least 16 weeks.

6. The regime/regimen of claim 1 or 2 wherein said composition comprises 0.001 to 2% adapalene by weight.

7. The regime/regimen of claim 1 or 2 wherein said composition comprises 0.1% adapalene by weight.

8. The regime/regimen of claim 1 or 2 wherein said composition comprises 0.3% adapalene by weight.

9. A regime/regimen for treating a patient afflicted with acne vulgaris which comprises first orally administering to the patient an effective amount of an antibacterial agent and topically applying to the afflicted skin region of the patient a therapeutically effective amount of a first dermatological composition comprising a therapeutically effective amount of adapalene and a dermatological vehicle on a regular basis for at least 12 weeks; and afterwards applying to the afflicted skin region on a regular basis a therapeutically effective amount of a second dermatological composition comprising a therapeutically effective amount of adapalene and a dermatological vehicle formulated as an aqueous gel, cream or lotion, without administering an antibacterial agent to the patient.

10. The regime/regimen of claim 9 wherein the oral antibacterial and the topical adapalene are administered every day of the first treatment period.

11. (canceled)

12. The regime/regimen of claim 9 wherein the antibacterial agent is selected from the group consisting of doxycycline, clindamycin, erythromycin, tetracycline, minocycline, trimethroprim, cotrimoxasole, limecycline, and benzoyl peroxide.

13. The regime/regimen of claim 9 wherein the second dermatological composition is applied daily or every other day.

14. The regime/regimen of claim 9 wherein the second dermatological composition is applied for at least 16 weeks.

15. The regime/regimen of claim 9 wherein the second dermatological composition comprises 0.001 to 2% adapalene by weight.

16. The regime/regimen of claim 9 wherein the second dermatological composition comprises 0.1% adapalene by weight.

17. The regime/regimen of claim 9 wherein the second dermatological composition comprises 0.3% adapalene by weight.

18. A regime/regimen for inhibiting the recurrence of acne vulgaris in a patient in which the clinical condition associated with acne vulgaris have been alleviated which comprises applying to the skin of said patient a topical composition comprising a therapeutically effective amount of adapalene and a topical vehicle therefor, without the administration of an antibacterial agent.

19. The regime/regimen of claim 18 wherein the topical composition is applied daily.

20. The regime/regimen of claim 18 wherein the topical composition is applied every other day.

21. The regime/regimen of claim 18 wherein the topical composition is applied for at least 16 weeks.

22. The regime/regimen of claim 18 wherein the topical composition comprises 0.001 to 2% adapalene by weight.

23. The regime/regimen of claim 18 wherein the topical composition comprises 0.1% adapalene by weight.

24. The regime/regimen of claim 18 wherein the topical composition comprises 0.3% adapalene by weight.
